(19) United States
(12) Patent Application Publication
(10) Pub. No.: US 2026/0031217 A1
(43) Pub. Date: Jan. 29, 2026

(54) METHOD AND APPARATUS FOR EMOTIONAL SELF-REPORTING

(71) Applicant: ELECTRONICS AND TELECOMMUNICATIONS RESEARCH INSTITUTE, Daejeon (KR)

(72) Inventors: Yong Ho LEE, Daejeon (KR); Cho Rong YU, Daejeon (KR); Sung Jin HONG, Daejeon (KR); Youn Hee GIL, Daejeon (KR); Ho Won KIM, Seoul (KR); Seong Min BAEK, Daejeon (KR)

(21) Appl. No.: 19/274,830

(22) Filed: Jul. 21, 2025

(30) Foreign Application Priority Data
Jul. 29, 2024 (KR) 10-2024-0100402
Jul. 17, 2025 (KR) 10-2025-0096826

(51) Int. Cl. G16H 20/70; G06T 3/4007

(52) U.S. Cl. CPC G16H 20/70; G06T 3/4007

(57) ABSTRACT

A method and apparatus for emotional self-reporting are disclosed. The method includes: continuously exploring an emotional state of a user within a PAD model space defined by X, Y, and Z axes using an input unit; receiving the explored emotional state as respective coordinate values of the of the X, Y, and Z axes; determining a final facial expression or a final emotional color corresponding to the emotional state of the user by integrating the coordinate values of the respective X, Y, and Z axes; and outputting, in real time, one or more of the final facial expression or the final emotional color using an output unit, wherein the X axis represents pleasure, the Y axis represents arousal, and the Z axis represents dominance, and each of the axes represents the emotional state using a scale defined by coordinate values ranging from −1 to +1.

### CROSS-REFERENCE TO RELATED APPLICATIONS

This application claims the benefit of and priority to Korean Patent Application No. 10-2024-0100402, filed Jul. 29, 2024, and Korean Patent Application No. 10-2025-0096826, filed Jul. 17, 2025, the entire disclosures of which are hereby incorporated herein by reference in their entirety.

### BACKGROUND OF THE INVENTION

### 1. Field of the Invention

The present disclosure relates to a method and apparatus for emotional self-reporting.

### 2. Description of Related Art

The following description simply provides only the background information related to the present embodiment without configuring the related art.

Theories that define human emotional states are largely classified into the discrete emotion theory and the dimensional model. The discrete emotion theory explains that humans have an innate set of basic emotions, and it classifies emotions on the basis of happiness, sadness, anger, surprise, fear, and disgust, which are Ekman's six basic emotions. The dimensional model is an approach that represents human emotions not as discrete categories but as positions on continuous axes or dimensions. The Pleasure-Arousal-Dominance (PAD) model, which is a three-dimensional emotion model, is a psychological model that can structurally explain the similarities and differences among individual emotions by representing emotional states using positions in a coordinate space composed of three continuous axes.

Emotion self-report technology refers to a means or method that helps users assess and report their current emotional states on their own, and it is used in various fields such as psychology, education, and human-computer interaction (HCI).

### SUMMARY OF THE INVENTION

The present disclosure is directed to providing a method and apparatus for emotional self-reporting. Specifically, it can enhance the consistency or reliability of emotional self-reporting based on the user's subjective judgment.

The objects of the present disclosure are not limited to those particularly described hereinabove, and the above and other objects that the present disclosure can achieve will be clearly understood by those skilled in the art from the following detailed description.

According to at least one aspect, the present disclosure provides a method for emotional self-reporting, the method comprising: continuously exploring an emotional state of a user within a PAD model space defined by X, Y, and Z axes using an input unit; receiving the explored emotional state as respective coordinate values of the of the X, Y, and Z axes; determining a final facial expression or a final emotional color corresponding to the emotional state of the user by integrating the coordinate values of the respective X, Y, and Z axes; and outputting, in real time, one or more of the final facial expression or the final emotional color using an output unit, wherein the X axis represents pleasure, the Y axis represents arousal, and the Z axis represents dominance, and each of the axes represents the emotional state using a scale defined by coordinate values ranging from −1 to +1.

According to another aspect, the present disclosure provides an apparatus for emotional self-reporting, the apparatus comprising: at least one memory configured to store instructions; and at least one processor, wherein the at least one processor, by executing the instructions, performs: continuously exploring an emotional state of a user within a PAD model space defined by X, Y, and Z axes using an input unit; receiving the explored emotional state as respective coordinate values of the of the X, Y, and Z axes; determining a final facial expression or a final emotional color corresponding to the emotional state of the user by integrating the coordinate values of the respective X, Y, and Z axes; and outputting, in real time, one or more of the final facial expression or the final emotional color using an output unit, wherein the X axis represents pleasure, the Y axis represents arousal, and the Z axis represents dominance, and each of the axes represents the emotional state using a scale defined by coordinate values ranging from −1 to +1.

According to at least one embodiment of the present disclosure, it is possible to enhance the consistency and reliability of emotional self-reporting by performing PAD model-based emotional self-reporting using various interfaces.

The effects of the present disclosure are not limited to those mentioned above, and other effects not mentioned will be apparent to those of ordinary skill in the art from the above description.

### BRIEF DESCRIPTION OF THE DRAWINGS

FIG. 1 is a block diagram of an apparatus for emotional self-reporting according to an embodiment of the present disclosure.

FIG. 2 is a diagram for describing a PAD model according to an embodiment of the present disclosure.

FIG. 3 is a diagram for describing an interface according to an embodiment of the present disclosure.

FIG. 4A is a diagram for describing a method of exploring emotional states using a mouse according to an embodiment of the present disclosure.

FIG. 4B is a diagram for describing a method of exploring emotional states using a keyboard according to an embodiment of the present disclosure.

FIG. 4C is a diagram for describing a method of exploring emotional states using a touchscreen according to an embodiment of the present disclosure.

FIG. 4D is a diagram for describing a method of exploring emotional states using a controller that is used in a mixed reality device according to an embodiment of the present disclosure.

FIG. 4E is a diagram for describing a method of exploring emotional states using a hand-tracking device according to an embodiment of the present disclosure.

FIG. 5A is a diagram for describing facial expressions according to an embodiment of the present disclosure.

FIG. 5B is a diagram for describing emotional colors according to an embodiment of the present disclosure.

FIG. 5C is a diagram for describing multidimensional images according to an embodiment of the present disclosure.

FIG. 6 is a flowchart schematically illustrating a method for emotional self-reporting according to an embodiment of the present disclosure.

FIG. 7 is a diagram schematically illustrating the configuration of an exemplary computing device that can be used to implement the apparatus and method described in the present disclosure.

### DETAILED DESCRIPTION OF THE INVENTION

Hereinafter, some exemplary embodiments of the present disclosure will be described in detail with reference to the accompanying drawings. In the following description, like reference numerals preferably designate like elements, although the elements are shown in different drawings. Further, in the following description of some embodiments, a detailed description of known functions and configurations incorporated therein will be omitted for the purpose of clarity and for brevity.

Additionally, various terms such as first, second, A, B, (a), (b), etc., are used solely to differentiate one component from the other but not to imply or suggest the substances, order, or sequence of the components. Throughout this specification, when a part ‘includes’ or ‘comprises’ a component, the part is meant to further include other components, not to exclude thereof unless specifically stated to the contrary. The terms such as ‘unit’, ‘module’, and the like refer to one or more units for processing at least one function or operation, which may be implemented by hardware, software, or a combination thereof.

The following detailed description, together with the accompanying drawings, is intended to describe exemplary embodiments of the present invention, and is not intended to represent the only embodiments in which the present invention may be practiced.

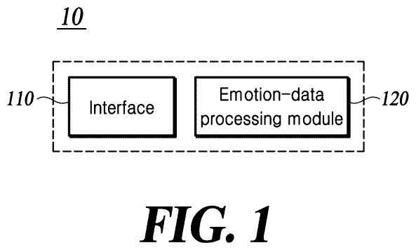

FIG. 1 is a block diagram of an apparatus for emotional self-reporting according to an embodiment of the present disclosure. Not all blocks shown in FIG. 1 are essential components, and in other embodiments, some blocks may be added, deleted, or modified. The components shown in FIG. 1 may be implemented as one or more software modules or components installed on one or more computing devices at one or more locations. In some implementations, one or more computing devices may be dedicated to specific components.

Hereafter, an apparatus for emotional self-reporting according to an embodiment of the present disclosure (10, hereafter referred to as a “self-reporting apparatus”) is described with reference to FIG. 1.

The self-reporting apparatus 10 is an apparatus that enables users to self-report their emotional states on the basis of a three-dimensional PAD model. The self-reporting apparatus 10 may include one or more of an interface 100 or an emotion-data processing module 120. The interface 110 can perform input and output functions for the user's emotion data. The emotion-data processing module 120 can perform functions such as visualization, weight calculation, storage, and transmission of emotion data.

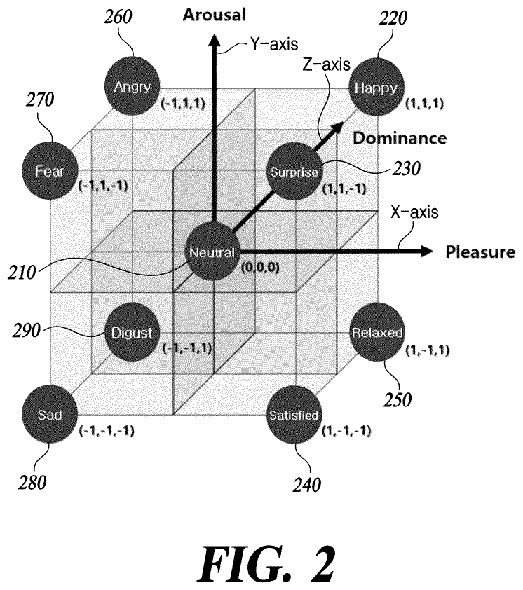

FIG. 2 is a diagram for describing a PAD model according to an embodiment of the present disclosure.

Hereafter, a PAD model that is used in the self-reporting apparatus 10 is described with reference to FIG. 2.

The self-reporting apparatus 10 uses a PAD model. In the PAD model, P represents pleasure, A represents arousal, and D represents dominance. They are represented using coordinate axes in the X, Y, and Z directions, respectively. The origin of the PAD model space is at coordinate (0, 0, 0), a scale ranging from −1 to +1 is defined for each axis, and emotional states can be represented as continuous numerical values. The positive values on the X-axis represent levels of pleasure and the negative values represent levels of displeasure. The positive values on the Y-axis represent levels of arousal and the negative values represent levels of calmness. The positive values on the Z-axis represent levels of dominance and the negative values represent levels of submissiveness. The scale can be defined as a continuous range set for numerically expressing or receiving emotional states.

The three-dimensional PAD model can be represented using a cube, and the center point and eight reference points are each defined with a corresponding emotion word. Referring to FIG. 2, on the basis of the coordinate values of the defined space, (0, 0, 0) corresponds to neutral 210, (1, 1, 1) corresponds to happy 220, (1, 1, −1) corresponds to surprise 230, (1, −1, −1) corresponds to satisfied 240, (1, −1, 1) corresponds to relaxed 250, (−1, 1, 1) corresponds to angry 260, (−1, 1, −1) corresponds to fear 270, (−1, −1, −1) corresponds to sad 280, and (−1, −1, 1) corresponds to disgust 290.

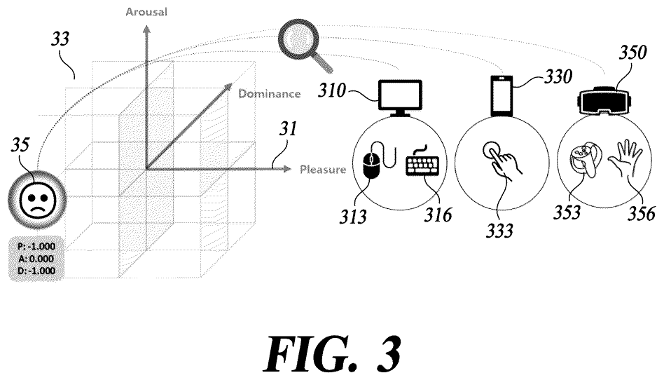

FIG. 3 is an exemplary diagram for describing an interface according to an embodiment of the present disclosure.

Hereafter, an interface that is used in the self-reporting apparatus 10 is described with reference to FIG. 3.

The interface 110 may include one or more of an output unit (not shown) or an input unit (not shown). The output unit is a component that visually provides a PAD model structure, coordinate values, facial expression images, color information, and the like in real time to intuitively output the user's emotional exploration and reporting processes. For example, the output unit may be implemented as a monitor 310, a touchscreen 330, a display of an MR (Mixed Reality) device, or a wearable display 350. The input unit is a unit for a user to select his or her emotional states on three-dimensional PAD model coordinates, and may be various physical or virtual input devices. For example, the input unit may be implemented as a mouse 313, a keyboard 316, a touchscreen 333, a controller 353 that is used in a mixed reality device, or a hand-tracking device 356 that recognizes hand gestures.

Referring to FIG. 3, the output unit can visually display the representation range of the PAD model in a 2D or 3D space on the basis of the X-, Y-, and Z-axes 31. The output unit can display the PAD model in a 2D or 3D space using a translucent or transparent cube 33. A user can explore his or her emotional state by freely moving in the PAD model space 31 or 33 using the input unit. The input unit may be a unit for receiving PAD coordinate values corresponding to emotional states from a user.

As a user freely explores the PAD model space 31 or 33 using the input unit, the user's emotional state 35 can be expressed and updated in real time. That is, the output unit can express the user's emotional state 35 in real time according to the corresponding coordinate values in the PAD model space 31 or 33.

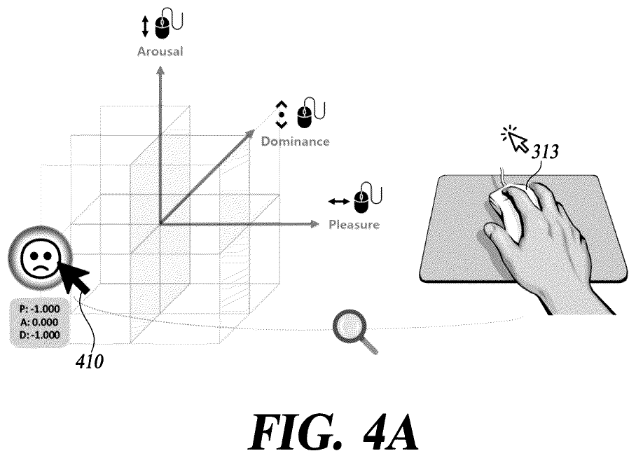

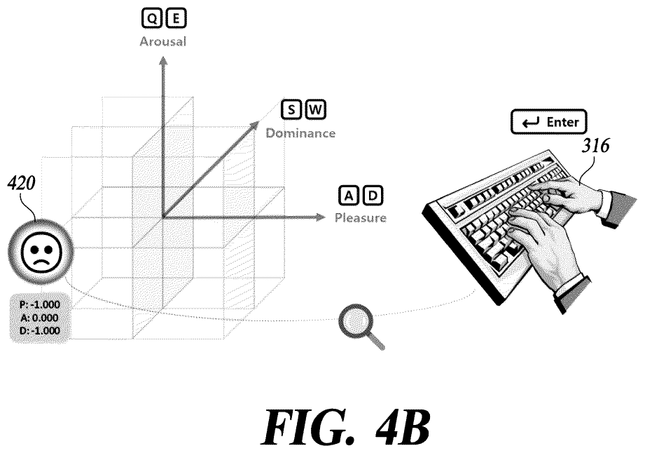

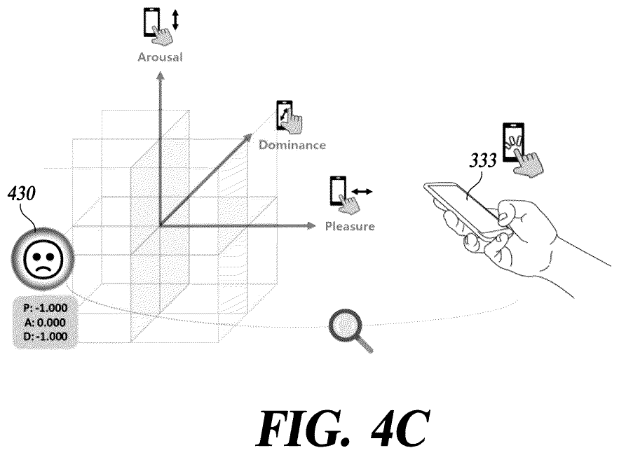

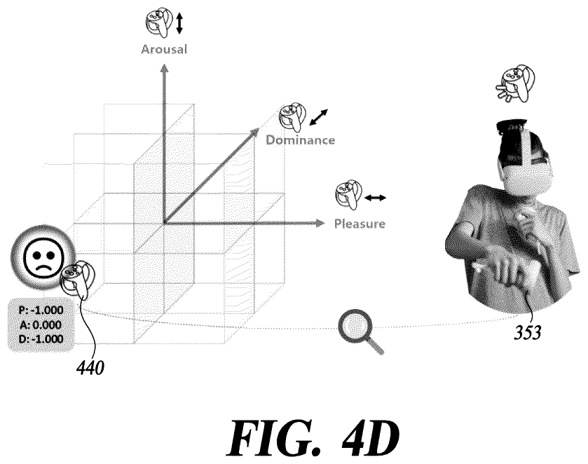

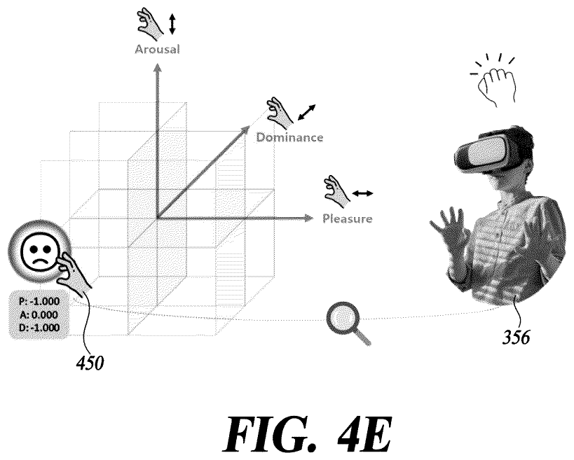

FIG. 4A is a diagram for describing a method of exploring emotional states using a mouse according to an embodiment of the present disclosure.

Referring to FIG. 4a, for example, a user can continuously explore the P dimension of the X-axis by moving the mouse 313 left and right, that is, along the horizontal axis, the A dimension of the Y-axis by moving the mouse 313 up and down, that is, along the vertical axis, and the D dimension of the Z-axis by scrolling the wheel of the mouse 313 up and down, that is, along the depth axis. During exploration, when the user determines that a coordinate value corresponds to his or her emotional state, the user can perform self-reporting by left-clicking the mouse 313. The user's real-time emotional state can be represented on the PAD model according to movement using the mouse pointer 410.

FIG. 4B is a diagram for describing a method of exploring emotional states using a keyboard according to an embodiment of the present disclosure.

Referring to FIG. 4b, for example, a user can explore emotional states using the keyboard 316. For horizontal movement along the P dimension of the X-axis, the button A can handle leftward movement and the button D can handle rightward movement. For vertical movement along the A dimension of the Y-axis, the button Q can handle downward movement and the button E can handle upward movement. For depth movement along the D dimension of the Z-axis, the button S can handle backward movement and the button W can handle forward movement. During exploration, when the user determines that a coordinate value corresponds to his or her emotional state, the user can perform self-reporting using the Enter button. The user's real-time emotional state 420 can be represented on the PAD model according to movement using the keyboard 316.

FIG. 4C is a diagram for describing a method of exploring emotional states using a touchscreen according to an embodiment of the present disclosure.

Referring to FIG. 4C, for example, a user can explore the P dimension of the X-axis by horizontally moving a finger on the touchscreen 333, and explore the A dimension of the Y-axis by vertically moving the finger. The user can explore the D dimension of the Z-axis by simultaneously touching the screen with two fingers and performing a pinch or spread gesture. During exploring, when the user determines that a coordinate value matches his or her emotional state, the user can submit a self-report using a touch gesture. The user's real-time emotional state 430 can be represented on the PAD model according to movement using the touchscreen 333.

FIG. 4D is a diagram for describing a method of exploring emotional states using a controller that is used in a mixed reality device according to an embodiment of the present disclosure.

Referring to FIG. 4D, for example, in the case of a content space provided by a head-mounted display (HMD), a user can explore a three-dimensional emotion model without dimensional reduction and can continuously explore the PAD model by freely moving the controller 353. During exploration, when the user determines that a coordinate value corresponds to his or her emotional state, the user can perform self-reporting using a trigger button of the controller 353. The user's real-time emotional state can be represented on the PAD model according to movement using a virtual object 440, such as a controller or a hand.

FIG. 4E is a diagram for describing a method of exploring emotional states using a hand-tracking device according to an embodiment of the present disclosure.

Referring to FIG. 4b, for example, a user can explore emotional states using the hand-tracking device 356. The user can continuously explore all dimensions of the PAD model by freely moving a three-dimensional space using a hand tracking device. During exploration, when the user determines that a coordinate value corresponds to his or her emotional state, the user can perform self-reporting by making a gesture of clenching and unclenching a fist. The user's real-time emotional state can be represented on the PAD model according to movement using a virtual hand 450.

The emotion-data processing module 120 can generate visual elements of emotional states on the basis of PAD coordinate values corresponding to the user's emotional states received from the interface 110.

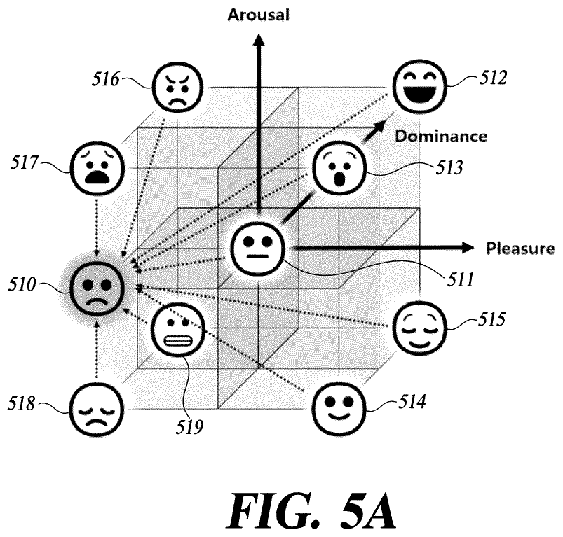

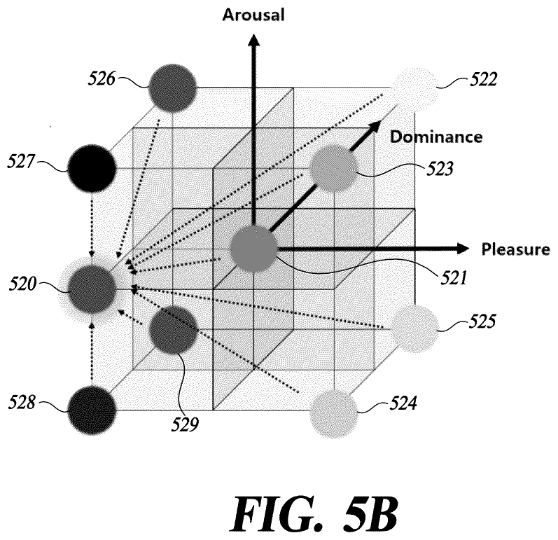

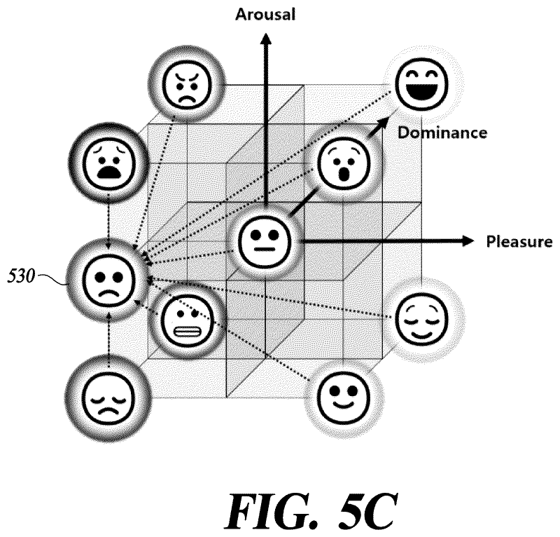

FIG. 5A is a diagram for describing facial expressions according to an embodiment of the present disclosure.

The emotion-data processing module 120 can generate a facial expression 510 that represents the current emotional state of a user. The facial expression 510 may be an emoji, a hand-drawn illustration, or a rendered avatar expression. The face can be represented as a male, female, or androgynous face. The emotion-data processing module 120 can generate in real time the facial expression 510 corresponding to the current emotional state of a user using an image morphing technique to which distance-based weight adjustment is applied, on the basis of facial expressions corresponding to emotional words corresponding to reference points.

For example, referring to FIG. 2 and FIG. 5a, facial expressions corresponding respectively to emotional words corresponding to reference points can be predefined, such as neutral 210 for 511 of FIG. 5a, happy 220 for 512 of FIG. 5a, surprise 230 for 513 of FIG. 5a, satisfied 240 for 514 of FIG. 5a, relaxed 250 for 515 of FIG. 5a, angry 260 for 516 of FIG. 5a, fear 270 for 517 of FIG. 5a, sad 280 for 518 of FIG. 5a, and disgust 290 for 519 of FIG. 5a.

An image morphing technique can use Equation 1.

$${w}_{i} = \frac{1}{{d}_{i}^{α}} \qquad [ Equation 1 ]$$

$$= \frac{{w}_{i}}{{\sum }_{j = 1}^{9} {w}_{j}}$$

$${I}_{p} = {{\sum }_{i = 1}}^{9} {I}_{i}$$

For an arbitrary point P=(x, y, z) in a three-dimensional space, when nine reference points are denoted as Vi, the weight wᵢ of each point can be defined as an inverse-distance weight. Here, dᵢ is the distance between P and Vi and α is a parameter for adjusting the weights and can be set as α=2. Subsequently, normalized weights [symbol] can be calculated to make the sum of all weights equal to 1. When the image of each reference point is denoted as Iᵢ, the image of a facial expression 510 corresponding to the emotional state of a user can be defined as Iₚ.

FIG. 5B is a diagram for describing emotional colors according to an embodiment of the present disclosure.

The emotion-data processing module 120 can generate an emotional color 520 that represents the current emotional state of a user. The emotion-data processing module 120 can generate in real time the emotional color 520 corresponding to the current emotional state of a user using an interpolation technique for RGB color values to which distance-based weight adjustment is applied, on the basis of emotional colors corresponding to emotional words corresponding to reference points.

For example, referring to FIG. 2 and FIG. 5b, emotional colors respectively corresponding to emotional words corresponding to reference points can be predefined, such as gray 521 for neutral 210, yellow 522 for happy 220, sky blue 523 for surprise 230, light green 524 for satisfied 240, mint 525 for comfortable 250, red 526 for angry 260, black 527 for fear 270, blue 528 for sad 280, and green 529 for disgust 290.

An interpolation (morphing) technique for RGB color values can use Equation 2.

$${C}_{P} = {{\sum }_{i = 1}}^{9} {C}_{i} \qquad [ Equation 2 ]$$

When the color of each reference point is Cᵢ (R, G, B), the emotional color 520 (indigo color) Cₚ corresponding to the emotional state of a user can be obtained on the basis of the normalized weight [symbol] obtained from Equation 1.

FIG. 5C is a diagram for describing multidimensional images according to an embodiment of the present disclosure.

Referring to FIG. 5C, the emotion-data processing module 120 can generate a multidimensional image 530 corresponding to the current emotional state of a user by combining the facial expression 510 and the emotional color 520 that correspond to the user's current emotional state. The multidimensional image 530 can intuitively express the user's current emotional state by showing the facial expression 510 and the emotional color 520, which correspond to the user's current emotional state, in real time and in parallel. Because the user can recognize his or her current emotional state in real time and intuitively, the reliability of emotional self-reporting can be improved.

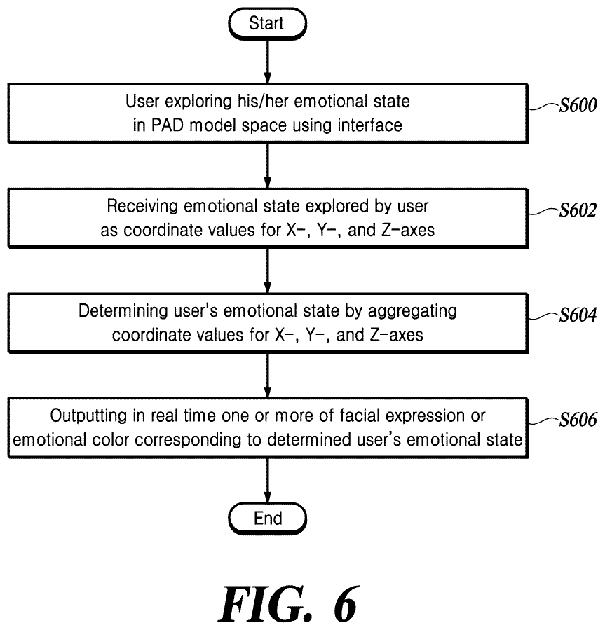

FIG. 6 is a flowchart schematically illustrating a method for emotional self-reporting according to an embodiment of the present disclosure.

A user can continuously explore the user's emotional state in the PAD model space 31 or 33 using the input unit 110 (S600). For example, the PAD model space may be a 2D or 3D space. For example, the input unit of the interface 110 may be one of a mouse 313, a keyboard 316, a touchscreen 333, a controller 353 of a mixed reality device, and a hand-tracking device 356 that recognizes hand gestures.

For example, the PAD model space composed of X-, Y-, and Z-axes may include reference points at which emotional words are respectively defined to mean neutral at (0, 0, 0), happy at (1, 1, 1), surprise at (1, 1, −1), satisfied at (1, −1, −1), comfortable at (1, −1, 1), angry at (−1, 1, 1), fear at (−1, 1, −1), sad at (−1, −1, −1), and disgust at (−1, −1, 1). For example, a facial expression or an emotional color that corresponds to the emotional word corresponding to each reference point can be predefined.

The emotion-data processing module 120 can receive the emotional state explored by the user as coordinate values for the X-, Y-, and Z-axes (S602).

The emotion-data processing module 120 can determine the user's emotional state by integrating the coordinate values for the X-, Y-, and Z-axes (S604). For example, using an image morphing technique to which distance-based weight adjustment is applied, the facial expression corresponding to the emotional state of a user can be determined on the basis of facial expressions respectively corresponding to the emotional words corresponding to reference points. For example, using an interpolation technique for color values to which distance-based weight adjustment is applied, the emotional color corresponding to the emotional state of a user can be determined on the basis of emotional colors respectively corresponding to the emotional words corresponding to reference points. For example, by combining the facial expression and the emotional color that correspond to the emotional state of a user, a multidimensional image corresponding to the user's emotional state can be determined.

The output unit of the interface 110 can output in real time one or more of the facial expression or the emotional color that corresponds to the determined user's emotional state (S606). For example, the output unit of the interface 110 may be one or more of a monitor 310, a touchscreen 330, a display of a mixed reality device, or a wearable display 350.

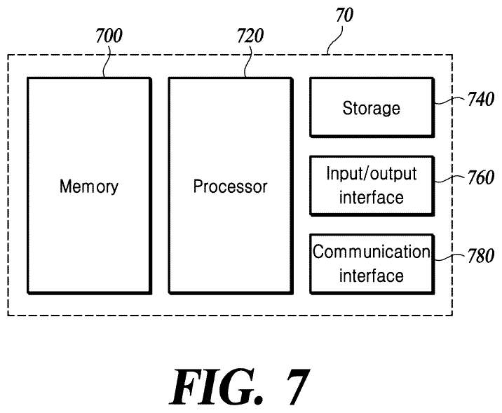

FIG. 7 is a diagram schematically illustrating the configuration of an exemplary computing device that can be used to implement the apparatus and method described in the present disclosure.

A computing device 70 may include some or all of a memory 700, a processor 720, a storage 740, an input/output interface 760, and a communication interface 780. The computing device 70 may be not only a stationary compute device such as a desktop computer and a server, but a mobile computing device such as a laptop computer and a smartphone. The computing device 70 may include any specialized hardware accelerator capable of efficiently processing computations for artificial intelligence models. For example, the computing device 70 may include a Graphic Processing Unit (GPU), a Tensor Processing Unit (TPU), or a Neural Processing Unit (NPU).

The memory 700 can store programs making the processor 720 perform methods or operations according to various embodiments of the present disclosure. For example, the program may include a plurality of instructions executable by the processor 720, and the methods or operations described above can be performed by executing the plurality of instructions through the processor 720. The memory 700 may be a single memory or a plurality of memories. In this case, the information for performing the methods or operations according to various embodiments of the present disclosure may be stored in a single memory or may be distributed across a plurality of memories. When the memory 700 is composed of a plurality of memories, the plurality of memories may be physically separated. The memory 700 may include at least one of a volatile memory and a nonvolatile memory. The volatile memory includes Static Random Access Memory (SRAM), Dynamic Random Access Memory (DRAM), or the like, and the nonvolatile memory includes flash memory or the like.

The processor 720 may include at least one core that can execute at least one instruction. The processor 720 can execute the instructions stored in the memory 700. The processor 720 may be a single processor or a plurality of processors.

The storage 740 retains stored data even if power supplied to the computing device 70 is interrupted. For example, the storage 740 may include nonvolatile memory and may also include storage media such as magnetic tape, optical disc, and magnetic disc. The programs stored in the storage 740 can be loaded into the memory 720 before being executed by the processor 700. The storage 740 can store files written in programming languages, and programs generated from the files by a compiler or the like can be loaded into the memory 700. The storage 740 can store data to be processed by the processor 720 and/or data that has been processed by the processor 720.

The input/output interface 760 may provide an interface with input devices such as a keyboard and a mouse and/or may include output devices such as a display device and a printer. A user can trigger execution of programs by the processor 720 through the input device and/or can check the processing results by the processor 720 through the output device.

The communication interface 780 can provide access to an external network. The computing device 70 can communicate with other devices through the communication interface 780.

The components described in the example embodiments may be implemented by hardware components including, for example, at least one digital signal processor (DSP), a processor, a controller, an application-specific integrated circuit (ASIC), a programmable logic element, such as an FPGA, other electronic devices, or combinations thereof. At least some of the functions or the processes described in the example embodiments may be implemented by software, and the software may be recorded on a recording medium. The components, the functions, and the processes described in the example embodiments may be implemented by a combination of hardware and software.

The method according to example embodiments may be embodied as a program that is executable by a computer, and may be implemented as various recording media such as a magnetic storage medium, an optical reading medium, and a digital storage medium.

Various techniques described herein may be implemented as digital electronic circuitry, or as computer hardware, firmware, software, or combinations thereof. The techniques may be implemented as a computer program product, i.e., a computer program tangibly embodied in an information carrier, e.g., in a machine-readable storage device (for example, a computer-readable medium) or in a propagated signal for processing by, or to control an operation of a data processing apparatus, e.g., a programmable processor, a computer, or multiple computers. A computer program(s) may be written in any form of a programming language, including compiled or interpreted languages and may be deployed in any form including a stand-alone program or a module, a component, a subroutine, or other units suitable for use in a computing environment. A computer program may be deployed to be executed on one computer or on multiple computers at one site or distributed across multiple sites and interconnected by a communication network.

Processors suitable for execution of a computer program include, by way of example, both general and special purpose microprocessors, and any one or more processors of any kind of digital computer. Generally, a processor will receive instructions and data from a read-only memory or a random access memory or both. Elements of a computer may include at least one processor to execute instructions and one or more memory devices to store instructions and data. Generally, a computer will also include or be coupled to receive data from, transfer data to, or perform both on one or more mass storage devices to store data, e.g., magnetic, magneto-optical disks, or optical disks. Examples of information carriers suitable for embodying computer program instructions and data include semiconductor memory devices, for example, magnetic media such as a hard disk, a floppy disk, and a magnetic tape, optical media such as a compact disk read only memory (CD-ROM), a digital video disk (DVD), etc. and magneto-optical media such as a floptical disk, and a read only memory (ROM), a random access memory (RAM), a flash memory, an erasable programmable ROM (EPROM), and an electrically erasable programmable ROM (EEPROM) and any other known computer readable medium. A processor and a memory may be supplemented by, or integrated into, a special purpose logic circuit.

The processor may run an operating system (OS) and one or more software applications that run on the OS. The processor device also may access, store, manipulate, process, and create data in response to execution of the software. For purpose of simplicity, the description of a processor device is used as singular; however, one skilled in the art will be appreciated that a processor device may include multiple processing elements and/or multiple types of processing elements. For example, a processor device may include multiple processors or a processor and a controller. In addition, different processing configurations are possible, such as parallel processors.

Also, non-transitory computer-readable media may be any available media that may be accessed by a computer, and may include both computer storage media and transmission media.

The present specification includes details of a number of specific implements, but it should be understood that the details do not limit any invention or what is claimable in the specification but rather describe features of the specific example embodiment. Features described in the specification in the context of individual example embodiments may be implemented as a combination in a single example embodiment. In contrast, various features described in the specification in the context of a single example embodiment may be implemented in multiple example embodiments individually or in an appropriate sub-combination. Furthermore, the features may operate in a specific combination and may be initially described as claimed in the combination, but one or more features may be excluded from the claimed combination in some cases, and the claimed combination may be changed into a sub-combination or a modification of a sub-combination.

Similarly, even though operations are described in a specific order on the drawings, it should not be understood as the operations needing to be performed in the specific order or in sequence to obtain desired results or as all the operations needing to be performed. In a specific case, multitasking and parallel processing may be advantageous. In addition, it should not be understood as requiring a separation of various apparatus components in the above described example embodiments in all example embodiments, and it should be understood that the above-described program components and apparatuses may be incorporated into a single software product or may be packaged in multiple software products.

It should be understood that the example embodiments disclosed herein are merely illustrative and are not intended to limit the scope of the invention. It will be apparent to one of ordinary skill in the art that various modifications of the example embodiments may be made without departing from the spirit and scope of the claims and their equivalents.

Accordingly, one of ordinary skill would understand that the scope of the claimed invention is not to be limited by the above explicitly described embodiments but by the claims and equivalents thereof.

What is claimed is:

1. A method for emotional self-reporting, the method comprising:
continuously exploring an emotional state of a user within a PAD model space defined by X, Y, and Z axes using an input unit;
receiving the explored emotional state as respective coordinate values of the of the X, Y, and Z axes;
determining a final facial expression or a final emotional color corresponding to the emotional state of the user by integrating the coordinate values of the respective X, Y, and Z axes; and
outputting, in real time, one or more of the final facial expression or the final emotional color using an output unit,
wherein the X axis represents pleasure, the Y axis represents arousal, and the Z axis represents dominance, and each of the axes represents the emotional state using a scale defined by coordinate values ranging from −1 to +1.

2. The method of claim 1, wherein the PAD model space defined of X, Y, and Z axes includes reference points at which emotional words are respectively defined to mean neutral at (0, 0, 0), happy at (1, 1, 1), surprise at (1, 1, −1), satisfied at (1, −1, −1), comfortable at (1, −1, 1), angry at (−1, 1, 1), fear at (−1, 1, −1), sad at (−1, −1, −1), and disgust at (−1, −1, 1).

3. The method of claim 2, wherein each reference point has a facial expression predefined to correspond to the emotional word of the reference point, and the final facial expression is determined, on the basis of the facial expressions corresponding to the emotional words, by using an image-morphing technique to which distance-based weighting is applied.

4. The method of claim 2, wherein each reference point has a emotional color predefined to correspond to the emotional word of the reference point, and the final emotional color is determined, on the basis of the emotional colors corresponding to the emotional words, by using an interpolation technique for color values to which distance-based weighting is applied.

5. The method of claim 1, wherein the PAD model space is a 2D space,
the input unit is one or more of a mouse, a keyboard, a touchscreen, a controller of a mixed reality device, or a hand-tracking device that recognizes hand gestures, and
the output unit is one or more of a monitor, a touchscreen, a display of a mixed reality device, or a wearable display.

6. The method of claim 1, wherein the PAD model space is a 3D space,
the input unit is one or more of a mouse, a keyboard, a touchscreen, a controller of a mixed reality device, or a hand-tracking device that recognizes hand gestures, and
the output unit is one or more of a monitor, a touchscreen, a display of a mixed reality device, or a wearable display.

7. An apparatus for emotional self-reporting, the apparatus comprising:
at least one memory configured to store instructions; and
at least one processor,
wherein the at least one processor, by executing the instructions, performs:
continuously exploring an emotional state of a user within a PAD model space defined by X, Y, and Z axes using an input unit;
receiving the explored emotional state as respective coordinate values of the of the X, Y, and Z axes;
determining a final facial expression or a final emotional color corresponding to the emotional state of the user by integrating the coordinate values of the respective X, Y, and Z axes; and
outputting, in real time, one or more of the final facial expression or the final emotional color using an output unit,
wherein the X axis represents pleasure, the Y axis represents arousal, and the Z axis represents dominance, and each of the axes represents the emotional state using a scale defined by coordinate values ranging from −1 to +1.

8. The apparatus of claim 7, wherein the PAD model space defined of X, Y, and Z axes includes reference points at which emotional words are respectively defined to mean neutral at (0, 0, 0), happy at (1, 1, 1), surprise at (1, 1, −1), satisfied at (1, −1, −1), comfortable at (1, −1, 1), angry at (−1, 1, 1), fear at (−1, 1, −1), sad at (−1, −1, −1), and disgust at (−1, −1, 1).

9. The apparatus of claim 8, wherein each reference point has a facial expression predefined to correspond to the emotional word of the reference point, and the final facial expression is determined, on the basis of the facial expressions corresponding to the emotional words, by using an image-morphing technique to which distance-based weighting is applied.

10. The apparatus of claim 8, wherein each reference point has a emotional color predefined to correspond to the emotional word of the reference point, and the final emotional color is determined, on the basis of the emotional colors corresponding to the emotional words, by using an interpolation technique for color values to which distance-based weighting is applied.

11. The apparatus of claim 7, wherein the PAD model space is a 2D space,
the input unit is one or more of a mouse, a keyboard, a touchscreen, a controller of a mixed reality device, or a hand-tracking device that recognizes hand gestures, and
the output unit is one or more of a monitor, a touchscreen, a display of a mixed reality device, or a wearable display.

12. The apparatus of claim 7, wherein the PAD model space is a 3D space,
the input unit is one or more of a mouse, a keyboard, a touchscreen, a controller of a mixed reality device, or a hand-tracking device that recognizes hand gestures, and
the output unit is one or more of a monitor, a touchscreen, a display of a mixed reality device, or a wearable display.
